SEQUENCE LISTING (7 sequences)

SEQ ID NO: 1 - protein, 34 aa, Conus geographus
ACSGRGSRCPPQCCMGLRCGRGNPQKCIGAHEDV

SEQ ID NO: 2 - protein, 48 aa, Agelenopsis aperta
EDNCIAEDYGKCTWGGTKCCRGRPCRCSMIGTNCECTPRLIMEGLSFA

SEQ ID NO: 3 - protein, 30 aa, Viola odorata
CAESCVYIPCTVTALLGCSCSNRVCYNGIP

SEQ ID NO: 4 - protein, 42 aa, Centruroides noxius
DRDSCVDKSRCAKYGYYQECQDCCKNAGHNGGTCMFFKCKCA

SEQ ID NO: 5 - protein, 40 aa, Lucilia sericata
ATCDLLSGTGVKHSACAAHCLLRGNRGGYCNGRAICVCRN

SEQ ID NO: 6 - protein, 52 aa, Scolopendra mutilans
TDDESSNKCAKTKRRENVCRVCGNRSGNDEYYSECCESDYRYHRCLDLLRNF

SEQ ID NO: 7 - protein, 18 aa, Artificial Sequence
  theta-defensin HTD-2 (retrocyclin 2)
GICRCICGRRICRCICGR

(19) United States
(12) Patent Application Publication
(10) Pub. No.: US 2017/0327891 A1
(43) Pub. Date: Nov. 16, 2017

(54) SYSTEM AND METHOD FOR IDENTIFYING PEPTIDE SEQUENCES

(71) Applicant: BAYLOR UNIVERSITY, Waco, TX (US)

(72) Inventors: Erich J. Baker, Waco, TX (US); Christopher Kearney, Woodway, TX (US); S. M. Ashiqul Islam, Waco, TX (US); Tanvir Sajed, Edmonton (CA)

(21) Appl. No.: 15/473,004

(22) Filed: Mar. 29, 2017

Related U.S. Application Data
(60) Provisional application No. 62/315,078, filed on Mar. 30, 2016.

(51) Int. Cl. C12Q 1/68; G01N 33/50; G06K 9/62; G06F 19/00

(52) U.S. Cl. CPC C12Q 1/6883; G06K 9/6269; G01N 33/5082; G06F 19/00

(57) ABSTRACT

A system and method for searching published genomes utilizes a robust and sensitive model to identify peptides that may serve as protein toxins. The protein toxins include unique cysteine stabilized structures and may be referred to as sequential tri-disulfide peptides (STPs) or as non-sequential tri-disulfide peptides (NTPs). While the sequence variability of STPs is so great that there are severe limitations to searching using traditional sequence-based methods, the present system and method efficiently and accurately identifies STPs as well NTPs from published genome databases, or in any peptide sequence, including artificial sequences.

This application claims priority to U.S. Provisional Patent Application Ser. No. 62/315,078, filed Mar. 30, 2016, entitled “System and Method for Identifying Peptide Sequences,” the entire content of which is hereby incorporated by reference.

### BACKGROUND

The present disclosure relates to a system and method for identifying or confirming peptide sequences of interest, particularly sequences for stable protein toxins.

Numerous organisms produce stable protein toxins that serve as antimicrobial peptides (AMPs) for use in attack (e.g., venoms) and self-defense (e.g., plant insecticides). These proteins are known to be toxic to living organisms and this toxicity can serve to provide defense for the host organism against opportunistic insects and microorganisms. In medicine and agriculture, naturally occurring toxic proteins provide an alternative to the rapidly dwindling supply of effective synthetic chemical insecticides, antimicrobials and antifungals. Naturally occurring toxic proteins have already been developed by traditional methods as bio-insecticides and antimicrobial peptides, and as scaffolds for drug discovery. The potential to search the genome for these toxins would greatly accelerate drug development beyond a traditional one-at-a-time approach. For example, if one wanted to develop an antimicrobial peptide for oral delivery, a search for these toxins in the published gut transcriptomes of insects or mammals would provide numerous strong leads. Niche applications would be possible, such as the creation of a dog-friendly snail bait by searching in a published green alga genome, since these algae are the natural prey of aquatic snails.

While there is tremendous value in searching for and identifying naturally occurring toxic proteins within published genomes, the toxins themselves share very little sequence identity. The sequence variability of the toxins is so great that there are severe limitations to searching using BLAST or other sequence-based algorithms. Thus, discovery has been slow and almost exclusively based on functional properties. The wide sequence variation, sources and modalities of group members impose serious limitations on the ability to rapidly identify potential members. As a result, there is a need for automated high-throughput member classification approaches that leverage their demonstrated tertiary and functional homology.

### SUMMARY

The present disclosure pertains to a system and method for identifying sequences for toxic peptides in peptide sequences.

Numerous organisms have evolved a wide range of toxic peptides for self-defense and predation. Their effective interstitial and macro-environmental use requires energetic and structural stability. For example, the physiological environment of an organism contains proteases and highly variable pH which can greatly impact peptide integrity. While a number of approaches can increase the stability of peptides under adverse environments, the inclusion of disulfide bonds is one natural way to increase stability. Stability through the inclusion of disulfide bonds is found in cystine stabilized toxins.

Despite a wide range of diversity based on their sources and modes of actions, all cystine stabilized toxins contain a fold with multiple disulfide connectivity. A sequential array of tri-disulfide connectivity is regarded as the most stable. A large number of these peptides include a sequentially paired disulfide bonding pattern (C1-C4, C2-C5, C3-C6), confirming a compact array of this cystine trio which is referred to herein as Sequential Tri-disulfide Peptides (STP). There may be other cysteines in the primary sequence of these peptides, but they do not participate in that sequential tridisulfide connectivity. This class of proteins includes several large protein families such as the well-defined knottins and cyclotide groups that have knotted tertiary structures, as well as scorpion toxin-like superfamily and a substantial proportion of diverse peptides comprising antimicrobial peptides and defensins. They also include a large number of stable toxins that contain the STP bonding pattern but lack the knotted motif typically created by C3-C6 in knottins and cyclotides.

For clarity, toxic peptides containing this particular stable disulfide connectivity can be referred to as sequential tri-disulfide peptide toxins (“STP toxins”). Going beyond these groupings, there are other stable toxins that exhibit compact tri-disulfide bonding patterns, but not in the sequentially paired model, including ladder-type toxins. Cystine stabilized toxins which do not contain the exact STP bonding array may also offer stability and toxicity and can be denoted as nonsequential tri-disulfide peptides (NTPs). FIG. 1 shows diagrams of disulfide connectivity of sequential tri-disulfide peptide toxins (STPs) as well as nonsequential tri-disulfide peptide toxins (NTPs). While STP toxins imply a compact tri-disulfide tertiary confirmation, NTPs toxins may contain both compact or non-compact tri-disulfide folds.

FIG. 2 shows a comparison of the compactness of disulfide bonds in different tri-disulfide array containing peptides. Distances are illustrated among the non-pairing sulfur molecules participating in the tri-disulfide array. Distances between different sulfur molecule pairs (balls) were measured using jmol software. The mean of these distances indicates the average distance among the disulfide bonds demonstrating the compactness of the tri-disulfide fold in the peptide. FIGS. 2(a), 2(b), 2(c), and 2(d) show distances of a sample representative of knotted STPs, nonknotted STPs, compact NTPs and non-compact NTPs, respectively, together with their Protein Data Bank (PDB) ids. The average of distance in STP toxins (FIGS. 2(a) and 2(b)) is typically less than 0.85 nm, while it is more than 1.2 nm in other tri-disulfide peptides such as in some non-compact NTPs such as those in FIG. 2(d). As seen in FIG. 2(c), some NTPs demonstrate a similar compactness (average distance) to STPs and can be designated as compact NTPs.

STP toxins can be further divided into three major groups based on their canonical 3D definitions: Cyclotides, inhibitor cystine knots (ICKs) and nonknotted STPs. Cyclotides form cyclization through N—C terminus adherence and are renowned as stable peptides containing the sequential tri-disulfide array. In this type of peptide, the third disulfide bond penetrates through the other two disulfide bonds participating in the array and forms a knotted macrocycle of disulfide bonds. ICKs, also known as knottins, are a second type of STPs. They contain the same knotted macrocycle as cyclotides but do not necessarily take the cyclic form. The third type has three sequentially paired disulfide bonds but the third bond does not penetrate the macrocycle, preventing the formation of a “knot.” This group may actually contain as many toxins as the first two subgroups combined and includes scorpion toxin-like peptides, insect peptides, plant peptides, and a variety of other peptides. All three STP subgroups are characterized by high stability and toxicity.

Traditional methods of searching for and discovering STP and NTP toxins has been hindered by their lack of sequence identify. In the case of ICKs, an automated discovery process based on sequence similarity using BLAST has previously been paired with sequence and structural algorithms (Knoter 1D and 3D, respectively) to precisely verify knottin candidates (Marsumura et al., 1989 and Gelly et al., 2004). The discovery of knottins via sequence similarity has produced an extensive and well-organized database, despite a scope limited to sequence similarity (Conibear et al., 2013). Cypred (Kedarisetti et al., 2014) is another relevant software that can predict cyclic proteins including those with STP like connectivity. While there is no known software to predict non-knotted STPs, there are databases focusing on limited specific families, such as CyBase for cyclotides (Mulvenna et al., 2006 and Wang et al., 2008), Conoserver for conotoxins (Kaas et al., 2012) and Arachnoserver for spider toxins (Herzig et al., 2011), but these have little broad application.

The present system and method overcome the limitations of traditional approaches and can efficiently and accurately identify STPs and NTPs from genome databases using unique feature sets. In particular, the system and method relates to a Support Vector Machine (SVM)-based model that predicts sequential tri-disulfide peptide (STP) toxins or nonsequential tri-disulfide peptide toxins (NTPs) from peptide sequences in a species-agnostic fashion. The ability to rapidly filter sequences for potential bioactive peptides can greatly compress the time between peptide identification and testing structural and functional properties for possible antimicrobial and insecticidal candidates.

In the present disclosure, a machine learning approach was utilized to reach a solution for the broad discovery of STP and NTP toxins through the use of soft or fuzzy classification schemas, based on salient STP or NTP features that extend beyond a reliance on primary sequence similarity. Logic-based machine learning has been used previously to classify the 2D structure of a/a domain type proteins (Muggleton et al., 1992), protein-protein interactions (Bock et al., 2001) or functional classifications of proteins from primary sequence. In particular, Support Vector Machines (SVM), a robust class of machine learning approaches (Cai et al., 2003), have been successfully used to predict cyclic proteins (Kedarisetti et al., 2014), 2D and 3D protein structures (Hua et al., J. Mol Biol. 2001 and Cai et al., 2001) and subcellular localization (Hua et al., Bioinformatics 2001) from primary sequence.

Knoter1D (Gracy et al., 2008) and Cypred (Kedarisetti et al., 2014) are examples of related software to discover cystine stabilized peptide toxins. Cypred is dedicated for detecting cyclic peptides. Knoter 1D is optimized to identify only knotted STPs using an algorithm that implements BLAST and is dependent on sequence identity with known knotted STPs. This approach does not allow Knoter1D to expand the inclusion of knotted STPs beyond a threshold of sequence identity. However, both knotted and non-knotted STPs vary in their sequences depending on the source organism.

After evaluating several feature sets, a combination of motif-based features and features based on individual amino acids (C, S, H, K, L) generated the best predictions, indicating that differentiation between STPs and nonSTPs lies in both inclusive motifs and primary sequences.

The present system and method pertain to a species-agnostic machine learning methodology, which in preferred embodiments may be referred to as PredSTP, which is designed to nominate undefined STPs or NTPs having low sequence identity with currently described STPs or NTPs. Efficient discovery of new functional members of this class of proteins will enhance the repertoire of potentially stable insecticidal and antimicrobial proteins. The present model was compared to existing approaches and demonstrates enhanced sensitivity and precision.

### BRIEF DESCRIPTION OF DRAWINGS

FIG. 1 shows diagrams of patterns of disulfide connectivity of different cysteine stabilized toxic peptides classified as (A) sequential tri-disulfide peptide toxins (STPs) and (B) nonsequential tri-disulfide peptide toxins (NTPs).

FIG. 2 shows diagrams illustrating the compactness of disulfide bonds in (A) knotted STPs, (B) nonknotted STPs, (C) compact NTPs, and (D) non-compact NTPs.

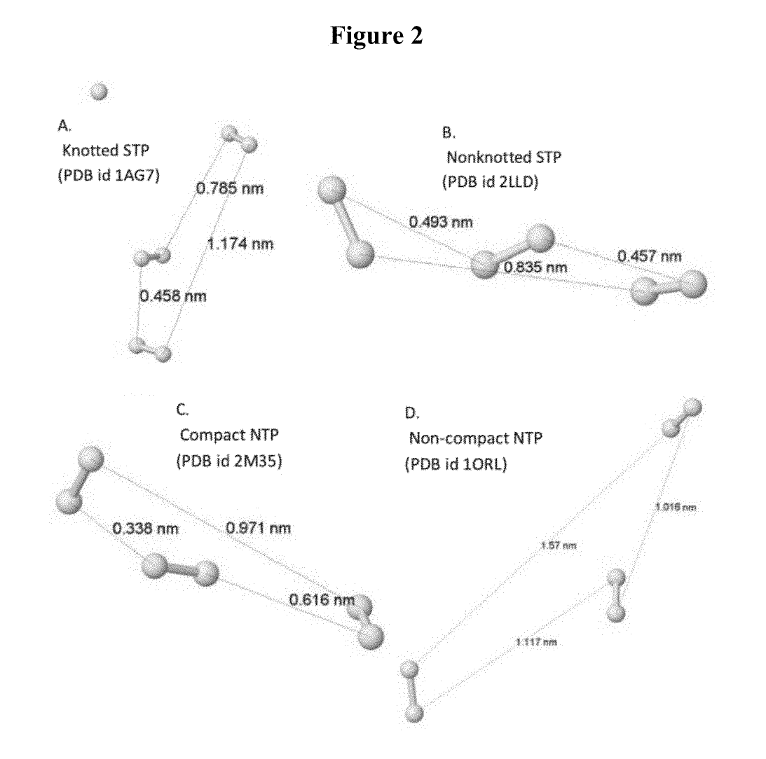

FIG. 3 shows a schematic of the process followed to develop and evaluate the model of the present disclosure.

FIG. 4 shows a distribution of size of the smallest loop length taken from a training set of sequences.

FIG. 5 shows a flow chart illustrating the calculation of proximity values for a sequence in a dataset.

FIG. 6 shows a flow chart illustrating the calculation of Normalized Bonding Distances (MBD) in a dataset.

FIG. 7 shows receiver operating characteristic (ROCR) curves for different tested feature sets.

FIG. 8 shows a flow chart illustrating the steps in analyzing training sequence data to identify STPs, in accordance with an exemplary embodiment.

FIG. 9 shows a flow chart illustrating the steps in analyzing an unknown sequence to predict whether it contains an STP, in accordance with an exemplary embodiment.

FIG. 10 shows a comparison of true positive hits for protein folds detected in a test set using different methods.

FIG. 11 shows a flow chart illustrating the steps in analyzing training sequence data to identify STPs or compact NTPs, in accordance with an exemplary embodiment.

FIG. 12 shows a flow chart illustrating the steps in analyzing an unknown sequence to predict whether it contains an STP or compact NTP, in accordance with an exemplary embodiment.

### DETAILED DESCRIPTION OF PREFERRED EMBODIMENTS

The present disclosure relates to a system and method for locating and identify sequential tri-disulfide peptide (STP) toxins and nonsequential tri-disulfide peptide (NTP) toxins. The system and method may be used for searching published genomes, or for analyzing synthetic or artificial sequences.

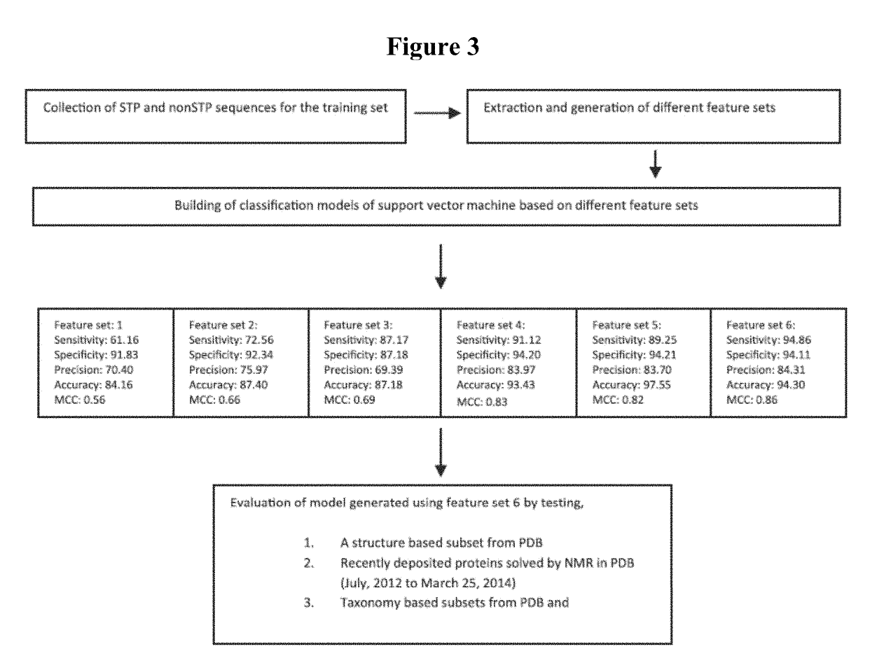

The present system and method utilize machine learning algorithms. It is imperative that successful machine learning algorithms select proper training sets and features. FIG. 3 shows a schematic of the process that was followed to develop and evaluate one embodiment of the SVM based STP and NTP toxin classifier that is the basis of the present system and method. A first step in this embodiment involved collection of STP and nonSTP sequences to be used for the training set.

First, for the known STP sequence collection, sequences of ICKs and cyclotides (knotted STPs) were collected from an available Knottin database (knottin.cbs.cnrs.fr/) and 167 sequences with solved 3D structures were obtained from this source. An additional 36 sequences of nonknotted STPs with known 3D structures were collected from Protein Data Bank (PDB) with 90% sequence identity (www.rcsb.org, June, 2013). The total set of 204 candidate sequences (167 from the knottin database and 37 from PDB) were further reduced to remove redundant sequences, defined as sequences sharing ≧90% sequence identity using CD-HIT (Huang et al., 2010 and Li, 2006). A total of 108 sequences were retained from the knottin database set and 36 sequences were from the PDB set, leaving 144 canonical STPs. The mean, standard deviation and range of the number of residues in the positive training set were 42.20, 15.70 and 23-143, respectively, with an average number of 6 cysteines per chain.

For the control negative sequence collection, sequences classified as negative control were collected from PDB using a criterion that was species agnostic and stipulated the exclusion of STPs through positive matches to PDB small proteins. This was defined as greater than 95% matches to the following PDB Query: “Experimental method is SOLUTION NMR; SCOP is small proteins; chain type: there is a protein chain but not any DNA or RNA or hybrid; stoichiometry in biological assembly: stoichiometry is MONOMER and TAXONOMY is Eukaryota (eucaryotes); released between 2000 and 2010.” Thus, the negative training set was constructed with a collection of small proteins verified from the NMR subset deposited in PDB between 2000 and 2010. They contain a similar number of total residues as STPs, and a number have tri-disulfide bonds (NTPs) in their 3D structure. 393 sequences were classified as non-STP sequences for the purposes of this disclosure. The mean, standard deviation and range of the number of residues in the chains of the negative training set are 63.16, 25.92 and 9-160, respectively, with an average number of 6 cysteines per chain.

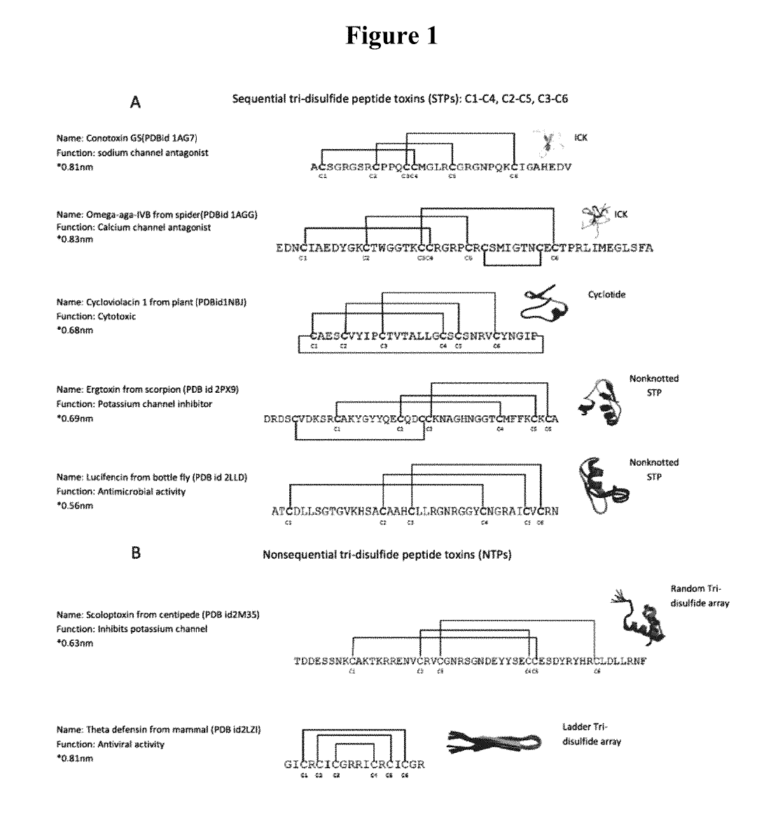

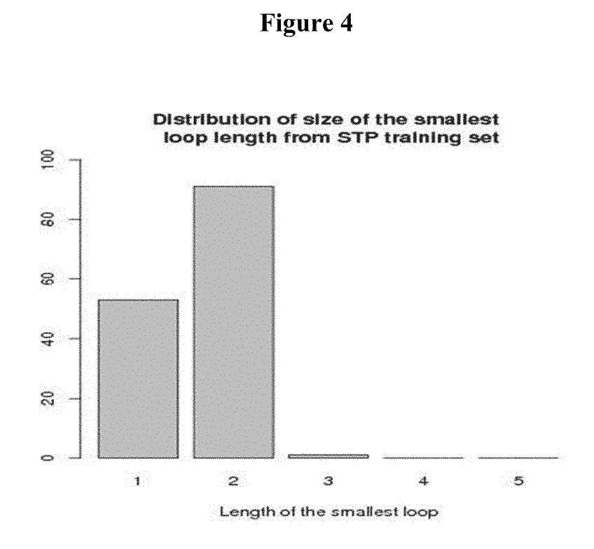

A next step in the process followed to develop and evaluate the present system and method, as shown in FIG. 3, involved the extraction and generation of different feature sets. All feature sets were created based on apparent characteristics as well as those offered by novel sequence evaluations, such as the Normalized Bonding Distance (NBD) between C1-C4, C2-C5 and C3-C6. Included in this step was defining the putative STP cystine motif. STP motifs consist of six cysteine residues (C1-C6) flanked by varying number of non-cysteine residues, as seen in FIG. 1. This set of consecutive cysteines was identified by elucidating the distance between each consecutive pair of cysteines, i and i+1 as ΔC_{i,i+1} (cysteine loops). First the sequences were searched for at least 6 cysteines in the query peptide sequence. If the sequence had at least six cysteines, then it calculated the distances between the consecutive cysteines. If the smallest distance was less than 3 residues the peptide was regarded as a candidate for STP motif (still not an STP candidate). By default the cysteines flanked by the smallest distance are considered as C3 and C4 in that motif. Thus, based on this global analysis of STP motifs, if the min(ΔC_{i,i+1}) was greater than three, then the motif was not considered to contain a STP and was discarded. FIG. 4 shows the distribution of size of the smallest loop lengths of control STP chains from the training set, with the most common length of the smallest loop being 1 or 2.

Likewise, if the min(ΔC_{i,i+1}) was less than or equal to three and located between C1 and C2 or C2 and C3 the motifs were disregarded as these motifs are often found within electron transport-like proteins such as ferredoxin, rubredoxin, and iron-sulfur proteins. Otherwise, the min(ΔC_{i,i+1}) was defined to exist between cysteines C3 and C4. This default pair of cysteines was shifted to a higher pair of cysteines if there existed less than 2 additional c-terminus cysteines. For example, if after the default C3 and C4 cysteines were identified, there was only one c-terminus cysteine, then the min(ΔC_{i,i+1}) was defined as cysteines C4 and C5. Also, if the smallest distance was between the first two or second two Cysteins in the primary sequence, then the STP motif was disregarded. So the cysteines in the STP motif were numbered according to their order in the motif, if the STP was not invalidated. There may be more than six cysteines in the peptide, but the order of cysteines was defined for those that were participating the putative STP motif. Other cysteines which were not participating in the STP motif were not given a particular order based identity (e.g., C1, C2, C3, C4, C5, C6).

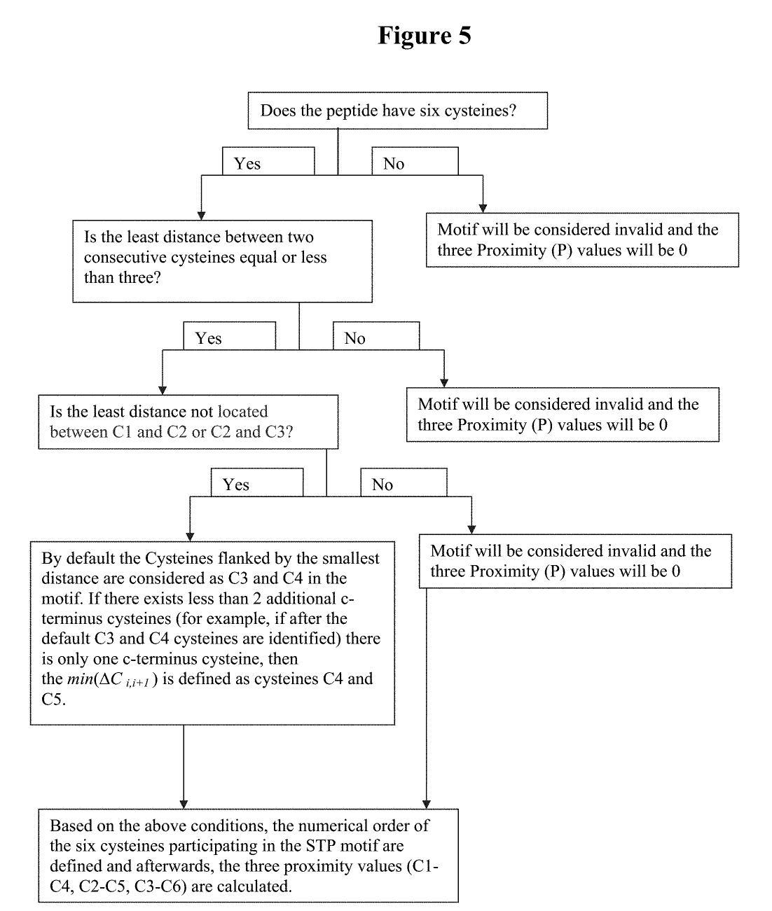

After putative STP motifs were identified and the order of each cysteine was defined in the STP motif, the Normalized Bonding Distances (NBD) were calculated. A set of three proximity lengths were calculated: P₁=ΔC_{1,4}; P₂=ΔC_{2,5}; P₃=ΔC_{3,6}. Motifs of less than six cysteines, or motifs defined as invalid by the utilized criteria, were assigned P₁=P₂=P₃=0. FIG. 5 shows a flow chart on calculation of these three proximity values.

A Normalized Proximity Length (NP) was then assigned for each proximity length, P, resulting in three new values: NP₁, NP₂, and NP₃. The NP identifies the distance from the observed mean proximity lengths of known STPs to the corresponding bonded cysteines involved in STP cysteine loops in the training set. For example, the average P for all STP sequences in the training set is subtracted from the calculated P value associated with its corresponding proximity length and normalized as shown in the equation below, where xPj is the average of the proximity lengths of known STPs derived from the training set.

NPjε{1,2,3}=1000/(|Pj−x̅Pj|+10)

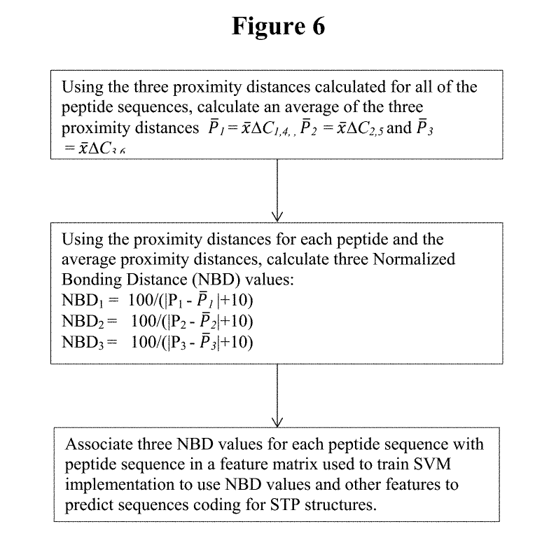

Here, if the query peptide doesn't have a true STP motif, all three P values will be zero and the NPj values will be something less than 10. On the other hand, if the three P values exactly equal to corresponding average P values for all the known STPs, then the P values will be exactly 10, which is the maximum value. So this step will generate three features providing the NP values for C1-C4, C2-C5 and C3-C6. FIG. 6 shows a flow chart on extraction of NP values, which may also be referred to as Normalized Bonding Distance (NBD) values.

Another feature utilized in the feature sets involved detecting the least loop length ratio. The least loop length is defined as the min(ΔC_{i,i+1}) divided by the total length of the peptide. This feature is used as part of feature sets 5 and 6, as shown below.

Another feature related to detecting the presence of amino acids between C4-C5 and C5-C6. Data published describing loop lengths of ICKs and cyclotides, which comprise a large subset of STPs, motivated a Boolean feature for the presence of interloop amino acids. A result of “true” was returned if there was a presence of a minimum of one amino acid in both of the last two loops (C4-C5 and C5-C6) in a putative STP motif.

Additional features were also considered in each of the tested feature sets, as shown below in Table 1. Overall, six unique sets of features were used in the machine learning protocol. The first feature set was derived from a multiple sequence alignment (MSA) using MUSCLE (Li, 2006) in MEGA 5.10. Here, each column was considered an independent feature, providing 318 unique features. Feature sets 2-6 were derived from a variety of sequence metadata, including composition and frequency of different amino acids, hydrophobicity, hydrophilicity, neutrality, bonding proximity score (defined below), total length of a chain and least loop to total length ratio (defined below), creating sets of 3, 23, 23, 28 and 28 features, respectively.

TABLE 1
Feature Sets

| Feature | No. of | |
|---|---|---|
| Set | Features | Features |
| 1 | 23 | Derived from calculating the frequency of |
| | | occurrence of each amino acid plus the frequency |
| | | of occurrence of aggregate hydrophobic (F, Y, L, |
| | | I, A, M, C, W, V), hydrophilic (R, K, N, D, A, P) |
| | | and neutral (G, H, S, T, Q) amino acids |
| 2 | 23 | Derived from calculating the number of |
| | | occurrences of each amino acid plus the |
| | | aggregate number of occurrences of |
| | | hydrophobic (F, Y, L, I, A, M, C, W, V), |
| | | hydrophilic (R, K, N, D, A, P), and |
| | | neutral (G, H, S, T, Q) amino acids |
| 3 | 3 | Derived from the Normalized Bonding Distance |
| | | (NBD) between C1-C4, C2-C5 and C3-C6 |
| 4 | 7 | Derived from the Normalized Bonding Distance |
| | | (NBD) between C1-C4, C2-C5 and C3-C6, |
| | | Presence of amino acid between C4-C5 and |
| | | C5-C6, presence of double consecutive cysteines |
| | | in the sequence in the 4^{th} and 5^{th} loop, total |
| | | peptide length and the least loop length ratio. The |
| | | latter was calculated by dividing the length of the |
| | | shortest ΔC_{i, i+1} by the total length of the peptide |
| 5 | 11 | Derived from Feature Set 4, plus calculating the |
| | | frequency of occurrences of cysteine, serine, |
| | | arginine, histindine, lysine (C, S, R, H, K) plus the |
| | | frequency of occurrences of hydrophobic (F, Y, |
| | | L, I, A, M, C, W, V), hydrophilic (R, K, N, D, |
| | | A, P), and neutral (G, H, S, T, Q) amino acids |
| 6 | 11 | Derived from Feature Set 4, plus calculating the |
| | | composition of cysteine, serine, arginine, histindine, |
| | | lysine (C, S, R, H, K) plus the aggregate number |
| | | of occurrences of hydrophobic (F, Y, L, I, A, |
| | | M, C, W, V), hydrophilic (R, K, N, D, A, P), and |
| | | neutral (G, H, S, T, Q) amino acids |

The next step in the process was the building of classification models of support vector machine based on different feature sets. A Support Vector Machine (SVM) classifier/predictor implementation was used to elucidate STP toxins. The SVM was implemented using the e1071 library in R (2.15.1).

The training data set of 144 STP and 393 non-STP chains was evaluated using randomized sampling of 100 and 300 random samples over 200 iterations to determine the optimal feature sets. All of the 6 feature sets were examined. Feature sets were assigned as described in Table 1 and sensitivity, specificity, precision and accuracy were determined after tenfold cross validation. Initial gamma and cost were set to 0.1 and 0.1, respectively, with the best output at 0.0587. A confusion matrix was created to perform the cross validation test. True Positives (TP), False Positives (FP), True Negatives (TN) and False Negatives (FN) were determined from the confusion matrix. Sensitivity [TP/(TP+FN)], specificity [TN/(TN+FP)], precision [TP/(TP+FP)], accuracy [(TP+TN)/(TP+FN+TN+FP)] and Mathews Correlation Coefficient (MCC) RTPXTN−FPXFN)/sqrt{(TP+FP)(TP+FN)(TN+FP)(TN+FN) were calculated to evaluate the performance of the algorithm.

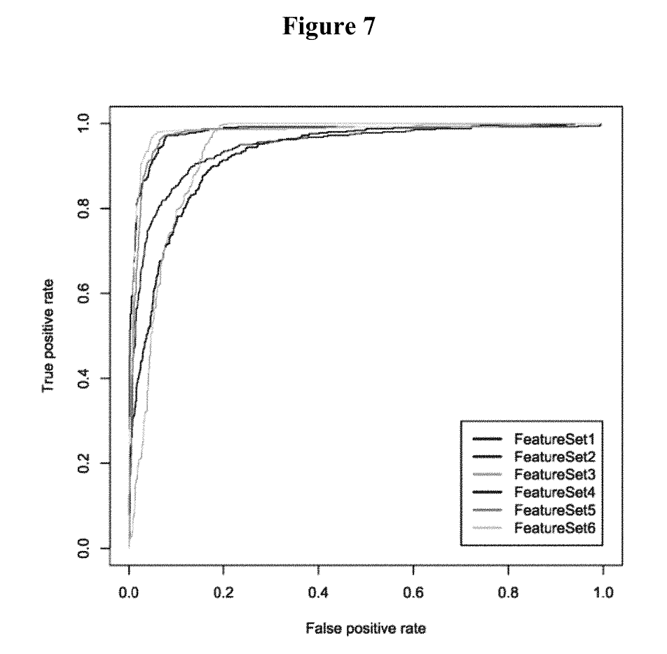

The sensitivity, specificity, precision, accuracy and MCC scores were calculated for each of the feature sets, and results are shown in FIG. 3. Feature set 6 demonstrated the best accuracy and MCC with values of 94.30%, and 0.86, respectively, and was selected as the best model. The Receptor Operating Curve (ROC) for feature set 6 is provided in FIG. 7. The area under curve (AUC) generated by feature set 1, 2, 3, 4, 5 and 6 were 0.84, 0.87, 0.87, 0.93, 0.92 and 0.94, respectively. The model represented by feature set 6 may be referred to herein as PredSTP.

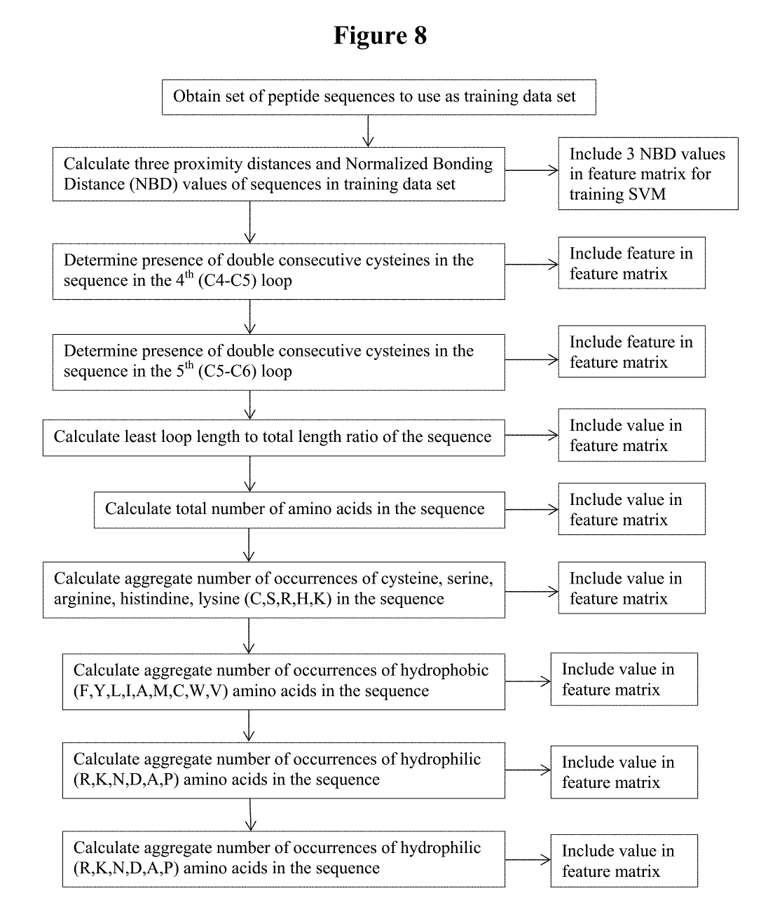

FIG. 8 shows a flow chart of an example of the model referred to as PredSTP, which is an exemplary embodiment of a method for searching a genome to identify sequences for STP toxins, and particularly shows the steps involved in preparing a feature matrix for use in training an SVM implementation. Generally, each time the PredSTP algorithm is used, a training data set is first processed to “train” the SVM implementation and to obtain the criteria by which each feature set is evaluated, which is included in a feature matrix later used for prediction. The training data set may be any appropriate number of peptides. In preferred embodiments, the training data set includes the training data set of 144 STP and 393 non-STP chains described herein. The precise criteria for each feature could vary depending on the training set used. However, the feature set used to train the SVM implementation preferably remains the same. The primary difference between the training data set and the unknown sequence is that the training data set also includes a confirmation of whether the sequence actually does contain a STP structure, which allows the SVM implementation to be trained to use the feature matrix to predict the presence of these structures in other sequences.

The first portion of the PredSTP model is illustrated in FIG. 5, showing how each peptide in the training set is analyzed and, for those having a valid STP motif, how the numerical order of cysteines in the STP motif are defined and three proximity distance values are calculated.

As shown in FIG. 6, in a next portion of the PredSTP model, Normalized Bonding Distance (“NBD”) is calculated from the three proximity distance values. The average of each of the three proximity distances is calculated for all of the peptides in the training set. These averages are calculated as follows: P̅₁=x̅ΔC_{1,4}, P̅₂=x̅ΔC_{2,5} and P̅₃=x̅ΔC_{3,6}. Using these average proximity distances, three normalized bonding distances are then calculated for each of the peptides in the training data set using the following equations:

NBD₁=100/(|P₁−P̅₁|+10)

NBD₂=100/(|P₂−P̅₂|+10)

NBD₃=100/(|P₃−P̅₃|+10)

The SVM implementation will consider the three calculated NBD values for all of the peptides in the training set and will determine a criteria by which an unknown sequence will be determined to have a STP structure based on an analysis of NBD values calculated for the unknown sequence as part of the model. The NBD values will be included in a feature matrix used to make the determination about the unknown sequence along with the other features calculated using the training data set.

FIG. 8 shows additional steps taken during the processing of the training data sets in order to evaluate features in the feature set. The peptides in the training data set are analyzed to determine the presence of double consecutive cysteines in the sequence in the 4^{th} (C4-C5) loop. If there are or are not double consecutive cysteines, this feature is designated as “True” or “False,” respectively, and these values are also included in the information used by SVM to evaluate the training data. In an additional similar step, the peptides in the training data set are analyzed to determine the presence of double consecutive cysteines in the sequence in the 5^{th} (C5-C6) loop. If there are or are not double consecutive cysteines, this feature is designated as “True” or “False,” respectively, and these values are also included in the information used by SVM to evaluate the training data.

FIG. 8 shows additional steps used with a training data set in the PredSTP model in which further features are evaluated. The first is the least loop length to total length of the peptide. This ratio is calculated as the min(ΔC_{i,i+1}) divided by the total length of the peptide. If the least loop length is more than 3, then the feature is considered as 0, which is interpreted by the SVM implementation as not contributing any likelihood that the peptide contains a STP structure. In addition to this least loop length ratio, the total number of amino acid residues of the protein sequence is calculated and included in the training set of feature data used by the SVM implementation.

FIG. 8 shows further steps in the PredSTP model used to train the SVM to screen for STP candidates. These steps include a calculation of each of the following: (1) The aggregate number of occurrences of cysteine, serine, arginine, histindine, lysine (C,S,R,H,K), (2) The aggregate number of occurrences of hydrophobic (F,Y,L,I,A,M,C,W,V) amino acids in the sequence, (3) The aggregate number of occurrences of hydrophilic (R,K,N,D,A,P) amino acids in the sequence, and (4) The aggregate number of occurrences of neutral (G,H,S,T,Q) amino acids in the sequence. For each of these, the SVM classifier/predictor implementation analyzes the peptides in the training data set, calculates these numbers, and associates them with a likelihood that a sequence will include a STP structure. Collectively, FIGS. 5-6 show how NBD values are calculated in an initial step of the PredSTP model, with an overall summary of the entire training process in this embodiment being presented in FIG. 8.

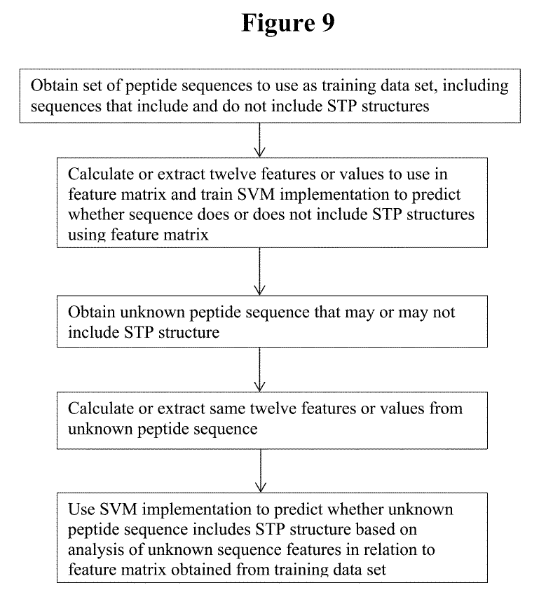

FIG. 9 shows the steps involved in screening an unknown peptide using the PredSTP model to determine if it contains an STP sequence. First, the training data set is analyzed as shown in greater detail in FIG. 8. Then, the unknown sequence is selected. Then, the same twelve features are extracted for the unknown sequence that were extracted for each training sequence: three NBD values, the presence of double consecutive cysteines in the C4-C5 loop, the presence of double consecutive cysteines in the C5-C6 loop, the least loop length to total length ratio, the total amino acid residues in the sequence, the aggregate number of occurrences of cysteine, serine, arginine, histindine, lysine (C,S,R,H,K), the aggregate number of occurrences of hydrophobic (F,Y,L,I,A,M,C,W,V) amino acids, the aggregate number of occurrences of hydrophilic (R,K,N,D,A,P) amino acids, and the aggregate number of occurrences of neutral (G,H,S,T,Q) amino acids. Then, using the feature matrix determined using the SVM implementation, the SVM analyzes the features of the unknown sequence and makes a prediction as to whether the unknown sequence contains a STP structure.

In preferred embodiments, a Support Vector Machine (SVM) classifier/predictor implementation is used to elucidate STP toxins. The SVM may preferably be implemented using the e1071 library in R (2.15.1). Sensitivity, specificity, precision and accuracy using the SVM may preferably be determined after ten-fold cross validation. Initial gamma and cost may be set to 0.1 and 0.1, respectively, and examples have demonstrated a best output at 0.0587. Given 144 STP and 393 non-STP chains used in a preferred embodiment, 100 and 300 random samples may be chosen, respectively, for a training set over 200 iterations.

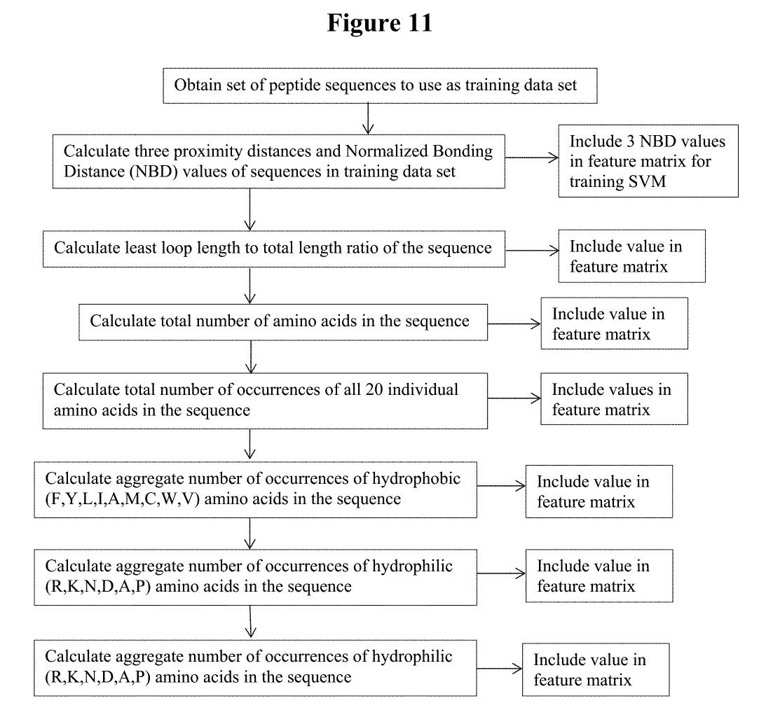

FIG. 11 shows a flow chart of another example of the model referred to as PredSTP, which is an exemplary embodiment of a method for searching a genome to identify sequences not just for STP toxins but also for NTPs, or those compact tri-disulfide peptides that have minor deviations from the sequential bonding pattern of C1-C4, C2-C5, C3-C6, but tend to have the same compact form as STPs. This embodiment of PredSTP can be used to detect any compact tri-disulfide stabilized peptide with features similar to STPs except for the sequential nature of the disulfide bonding. Again, each time the PredSTP algorithm is used, a training data set is first processed to “train” the SVM implementation and to obtain the criteria by which each feature set is evaluated, which is included in a feature matrix later used for prediction. In this embodiment, the training data set also includes sequences which are positively confirmed to contain NTPs, which allows the SVM implementation to be trained to use the feature matrix to predict the presence of these structures in other sequences. The features included in the feature matrix are similar to those set out for PredSTP used to predict STPs only, with a couple of changes.

Again, the first portion of the PredSTP model in this embodiment is illustrated in FIG. 5, showing how each peptide in the training set is analyzed and, for those having a valid motif (which could be STP or compact NTP), how the numerical order of cysteines in the motif are defined and three proximity distance values are calculated.

As shown in FIG. 6, in a next portion of the PredSTP model, Normalized Bonding Distance (“NBD”) is calculated from the three proximity distance values. The average of each of the three proximity distances is calculated for all of the peptides in the training set. These averages are calculated as follows: P̅₁=x̅ΔC_{1,4}, P̅₂=x̅ΔC_{2,5} and P̅₃=x̅ΔC_{3,6}. Using these average proximity distances, three normalized bonding distances are then calculated for each of the peptides in the training data set using the following equations:

NBD₁=100/(|P₁−P̅₁|+10)

NBD₂=100/(|P₂−P̅₂|+10)

NBD₃=100/(|P₃−P̅₃|+10)

The SVM implementation will consider the three calculated NBD values for all of the peptides in the training set and will determine a criteria by which an unknown sequence will be determined to have a STP or compact NTP structure based on an analysis of NBD values calculated for the unknown sequence as part of the model. The NBD values will be included in a feature matrix used to make the determination about the unknown sequence along with the other features calculated using the training data set.

FIG. 11 shows additional steps used with a training data set in this embodiment of the PredSTP model in which further features are evaluated. These are similar but not identical to those of the preferred embodiment relating to STP sequences. The first is the least loop length to total length of the peptide. This ratio is calculated as the min(ΔC_{i,i+1}) divided by the total length of the peptide. If the least loop length is more than 3, then the feature is considered as 0, which is interpreted by the SVM implementation as not contributing any likelihood that the peptide contains a STP structure. In addition to this least loop length ratio, the total number of amino acid residues of the protein sequence is calculated and included in the training set of feature data used by the SVM implementation.

FIG. 11 shows further steps in the PredSTP model used to train the SVM to screen for STP or compact NTP candidates. These steps include a calculation of each of the following: (1) The total number of occurrences of each of the individual twenty (20) amino acids in the sequence (which results in 20 different features), (2) The aggregate number of occurrences of hydrophobic (F,Y,L,I,A,M,C,W,V) amino acids in the sequence, (3) The aggregate number of occurrences of hydrophilic (R,K,N,D,A,P) amino acids in the sequence, and (4) The aggregate number of occurrences of neutral (G,H,S,T,Q) amino acids in the sequence. For each of these, the SVM classifier/predictor implementation analyzes the peptides in the training data set, calculates these numbers, and associates them with a likelihood that a sequence will include a STP or compact NTP structure. Collectively, FIGS. 5-6 show how NBD values are calculated in an initial step of the PredSTP model, with an overall summary of the entire training process of this embodiment being presented in FIG. 11. In this embodiment, a total of 28 features are extracted for the feature matrix.

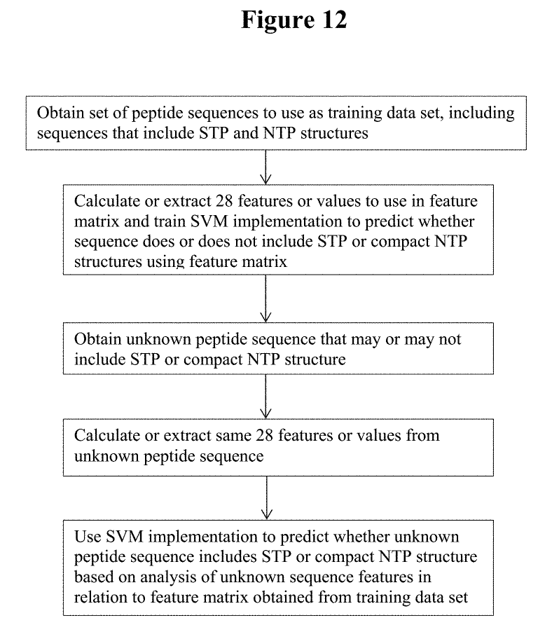

FIG. 12 shows the steps involved in screening an unknown peptide using the PredSTP model to determine if it contains an STP or compact NTP sequence. First, the training data set is analyzed as shown in greater detail in FIG. 11. Then, the unknown sequence is selected. Then, the same 28 features are extracted for the unknown sequence that were extracted for each training sequence: three NBD values, the least loop length to total length ratio, the total amino acid residues in the sequence, the total number of occurrences of all 20 individual amino acids in the sequence, the aggregate number of occurrences of hydrophobic (F,Y,L,I,A,M,C,W,V) amino acids, the aggregate number of occurrences of hydrophilic (R,K,N,D,A,P) amino acids, and the aggregate number of occurrences of neutral (G,H,S,T,Q) amino acids. Then, using the feature matrix determined using the SVM implementation, the SVM analyzes the features of the unknown sequence and makes a prediction as to whether the unknown sequence contains a STP or compact NTP structure.

The present system and method are particularly useful for searching genomes to locate potential STP and NTP structures within actual published sequences, but it should be noted that the system and method are equally applicable to analyzing synthetic or artificial sequences that may be created from scratch specifically to generate STP or NTP structures. Thus the PredSTP model is also directly applicable to the de novo in silico design of specialized proteins such as antimicrobial peptides (AMPs) and insecticides. Similarly, the algorithm is useful to ensure that, in the use of an STP (or NTP) as scaffold for drug design, any drugs built as variants of the scaffold would indeed stay within the structural boundaries of a canonical STP (or NTP).

### EXAMPLES

To evaluate the PredSTP model, independent test sequences were collected and considered. Table 2 below shows the eight independent sets of sequences that were collected to verify the robustness of the model.

TABLE 2

| Independent test | Query parameters | Number of | Number of |
|---|---|---|---|
| sample | (PDB^{a}) | proteins | chains |
| Small protein 92 | SCOP: Small Proteins | 92 | 163 |
| | Experimental Method: X-RAY | | |
| | Resolution: 1.499 or less | | |
| Only Eukaryote | TAXONOMY: Eukaryota | 45751 | 102748 |
| Only Bacteria | TAXONOMY: Bacteria | 31664 | 80664 |
| | (eubacteria) | | |
| Only Archaea | TAXONOMY: Archaea | 3127 | 8366 |
| Only Virus | TAXONOMY: Viruses | 4629 | 18642 |
| Unassigned | TAXONOMY: Unassigned | 479 | 980 |
| Others | TAXONOMY: Other sequences | 457 | 1418 |
| | or unclassified sequences | | |
| Recently deposited | Experimental Method: solution | 657 | 751 |
| proteins solved by | NMR | | |
| NMR in PDB (July | | | |
| 2012 to Mar. 25 2014) | | | |

^{a}PDB date August 2013 unless otherwise noted. Protein chain types only.

Among these were sets classified according to Protein Data Bank (PDB, July 2013) criteria as Eukaryote, Bacteria, Archaea, Virus and Unassigned. In addition, a set of proteins whose sequences were recently solved by NMR and deposited in PDB (Jul. 4, 2012 to Mar. 25, 2014) (“NewNMR751”) and also the Structural Classification of Protein (SCOP) PDB subset were used (“Smallprotein163”). Small protein sequences were retrieved with the following parameters: (a) resolution <1.5 Å, (b) protein chain but not DNA/RNA/Hybrid, and (c) limited to small disulfide rich proteins and have similarity in size, number of disulfide bonds, cystine number and cysteine arrangements in their primary structure. The result included STPs, rubredoxins, BPTI-like, snake toxin-like, crambin-like, insulin-like, and high potential iron proteins among others.

### Example 1. Predicting STP Sequences

STP sequences were predicted from certain of the test sets shown in Table 2 using feature set 6. Due to the limited throughput of the Knoter1D interface, only the predictions made using the “NewNMR751” and “Smallprotein163” sets defined above were compared against Knoter 1D predictions (knottin.cbs.cnrs.fr/Tools_1D.php) and validated with Jmol by analyzing the disulfide connectivity using the corresponding PDB files. Results from only the eukaryotic test sets were filtered to remove sequences with ≧30% chain identity and compared against Jmol analysis. Chains exhibiting canonical STP connectivity (C1-C4, C2-C5, C3-C6) were initially considered as true positives. True positives were further cross matched with their PDB annotations to make the final confirmation.

The SmallProtein163 data subset from PDB was analyzed to determine potential automated STP classification. The median residue number of the chains in the Smallprotein163 subset is 54, which is similar to the number of residues in STP chains. In addition, 94 out of the 163 chains contain at least 6 cysteines in their primary sequences. From this subset, PredSTP was able to identify 21 of the 163 potential chains as STPcontaining. These putative STP structures were verified by examining their disulfide bonding patterns in Jmol. Of the 21 identified chains by PredSTP, 14 of them were confirmed as true positives, as shown in Table 3 below.

TABLE 3

| Total PredSTP | TRUE | Knoter 1D |
|---|---|---|
| positive chains | positive | positive |
| 21 | 14/21 | 1/21 |

An analysis of the 142 negative STP chains predicted by PredSTP demonstrated only one false negative. The sensitivity, specificity, precision and accuracy for this particular dataset were 93.33%, 99.29%, 66.66% and 95.09%, respectively, as shown in Table 8 below. Table 4 below shows the list and description of the 21 proteins in the “Smallprotein163” subset positively predicted using PredSTP.

TABLE 4

| | Domain | | | |
|---|---|---|---|---|
| | stabilized by tri- | | | |
| PDB ID¹ | disulfide bonds | Disulfide connectivity | Knoter1D | Function/Class |
| *1AHO | Yes | (C1-C4, C2-C5, C3-C6) | No | Scorpion Neurotoxin |
| 1BX7 | Yes | Array is not | No | Serine Protease Inhibitor |
| | | compact or absent | | |
| 1BX8 | Yes | Array is not | No | Serine Protease Inhibitor |
| | | compact or absent | | |
| *1DJT: A | Yes | (C1-C4, C2-C5, C3-C6) | No | Alpha-like Neurotoxin |
| *1DJT: B | Yes | (C1-C4, C2-C5, C3-C6) | No | Alpha-like Neurotoxin |
| *1KV0: A | Yes | (C1-C4, C2-C5, C3-C6) | No | Alpha-like Toxin |
| *1KV0: A | Yes | (C1-C4, C2-C5, C3-C6) | No | Alpha-like Toxin |
| *1LU0: A | Yes | (C1-C4, C2-C5, C3-C6) | Yes | Hydrolase Inhibitor |
| *1LU0: B | Yes | (C1-C4, C2-C5, C3-C6) | Yes | Hydrolase Inhibitor |
| *1NPI | Yes | (C1-C4, C2-C5, C3-C6) | No | Neurotoxin |
| 1P9G | Yes | C1-C4, C2-C6, C3-C5 | No | Antifungal Protein |
| *1PTX | Yes | (C1-C4, C2-C5, C3-C6) | No | Scorpion Toxin |
| 1R0R | Yes | Array is not | No | Serine Protease |
| | | compact or absent | | |
| *1SEG | Yes | (C1-C4, C2-C5, C3-C6) | No | Scorpion Alpha Toxin |
| 1SGP | No | Array is not | No | Serine Protease/Inhibitor |
| | | compact or absent | | |
| *1SN4 | Yes | (C1-C4, C2-C5, C3-C6) | No | Scorpion Neurotoxin |
| *1T7E | Yes | (C1-C4, C2-C5, C3-C6) | No | Alpha-Like Neurotoxin |
| *2ASC | Yes | (C1-C4, C2-C5, C3-C6) | No | Scorpion Toxin |
| 2GKR | Yes | Array is not | No | Hydrolase Inhibitor |
| | | compact or absent | | |
| *2SN3 | Yes | (C1-C4, C2-C5, C3-C6) | No | Scorpion Neurotoxin |
| 2UUY | Yes | C1-C3, C2-C6, C4-C5 | No | Tryptase Inhibitor |

¹*= True positives

PredSTP was also tested against protein sequences with less than 90% sequence identity and recently solved (Jul. 4, 2012 to Mar. 25, 2014) by NMR. This set of 751 amino acid chains is denoted as NewNMR751 and has a median number of 82 residues with 118 chains containing more than six cysteines. The model detected 23 chains from 23 different proteins. Analyzing the disulfide connectivity of the positive hits by Jmol, 21 chains were confirmed as true positive. Based on the number of the predicted outcomes, the sensitivity, specificity, precision and accuracy for this particular dataset were 91.30%, 99.72%, 91.30% and 99.46%, respectively, as shown in Table 8 below.

The true positive chains were further classified into 9 ICKs, 5 cyclotides and 7 nonknotted STPs. PDB identifications and functions for positive predictions are shown in Table 5 below.

TABLE 5

| | | | | Predicted |
|---|---|---|---|---|
| | Functional | Disulfide connectivity | True | by |
| PDB id | classification | in the putative motif | positive | Knoter1D |
| *2LIX | Potassium channel | C1-C4, C2-C5, C3-C6 | Yes | No |
| | toxin | | | |
| *2LJ7 | Antimicrobial | C1-C4, C2-C5, C3-C6 | Yes | No |
| | Peptide | | | |
| *2LJS | Cyclotide | C1-C4, C2-C5, C3-C6 | Yes | Yes |
| *2LL1 | Spider toxin | C1-C4, C2-C5, C3-C6 | Yes | Yes |
| *2LN4 | Antimicrobial | C1-C4, C2-C5, C3-C6 | Yes | No |
| | Peptide | | | |
| *2LT8 | Antimicrobial | C1-C4, C2-C5, C3-C6 | Yes | No |
| | Peptide | | | |
| *2LU9 | Potassium channel | C1-C4, C2-C5, C3-C6 | Yes | No |
| | toxin | | | |
| *2LUR | Cyclotide | C1-C4, C2-C5, C3-C6 | Yes | Yes |
| *2LY5 | Defensin-like | C1-C4, C2-C5, C3-C6 | Yes | No |
| *2LZX | New ICK toxin from | C1-C4, C2-C5, C3-C6 | Yes | No |
| | sponge | | | |
| *2M2Q | New ICK toxin from | C1-C4, C2-C5, C3-C6 | Yes | No |
| | bitter melon | | | |
| *2M2R | New ICK toxin from | C1-C4, C2-C5, C3-C6 | Yes | No |
| | bitter melon | | | |
| *2M36 | Spider toxin | C1-C4, C2-C5, C3-C6 | Yes | Yes |
| 2M3H | Apoptotic protein | **Array is not | No | No |
| | | compact or absent | | |
| *2M3J | New ICK toxin from | C1-C4, C2-C5, C3-C6 | Yes | No |
| | sponge | | | |
| *2M4Z | Spider toxin | C1-C4, C2-C5, C3-C6 | Yes | Yes |
| *2M86 | Cyclotide | C1-C4, C2-C5, C3-C6 | Yes | Yes |
| *2M9O | Cyclotide | C1-C4, C2-C5, C3-C6 | Yes | Yes |
| 2MD7 | Transcription | **Array is not | No | No |
| | | compact or absent | | |
| *2MH1 | Cyclotide | C1-C4, C2-C5, C3-C6 | No | Yes |
| *4B2U | New ICK toxin | C1-C4, C2-C5, C3-C6 | Yes | No |
| | sicarius spiders | | | |
| *4B2V | New ICK toxin | C1-C4, C2-C5, C3-C6 | Yes | No |
| | sicarius spiders | | | |
| *4BMF | Hydrolase | C1-C4, C2-C5, C3-C6 | Yes | No |

This set was also analyzed by PSI BLAST (Altschul et al., 1997) and Knoter1D. For PSI BLAST, the BLAST suite (blast-2.2.29+) was installed on a local machine along with the appropriate dataset. The dataset was the NewNMR751 dataset. The selected threshold e-values for PSI BLAST were 0.01, 0.1 and 0.5. The number of iterations for PSI BLAST was 5. All other parameters were set as default.

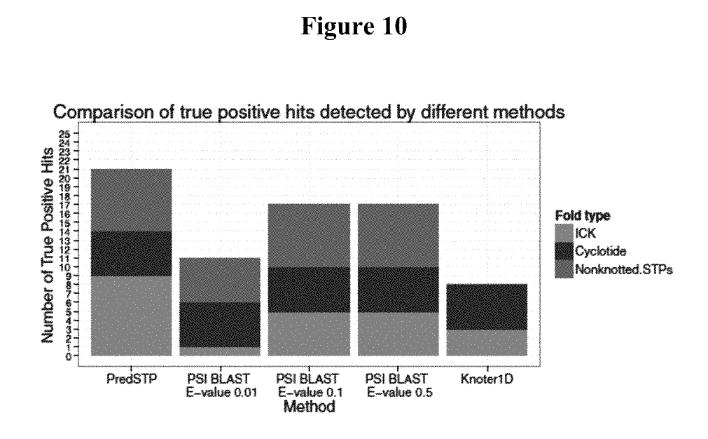

Knoter1D detected 5 cyclotides, 3 of the 9 ICKs and none of the nonknotted STPs. PSI BLAST (e-value 0.01) detected 12 chains comprising 1 ICK, 5 cyclotides, 5 nonknotted STPs and 1 false positive. PSI BLAST (e-value 0.1) detected 21 chains comprising five ICK, five cyclotides, seven nonknotted STPs and four false positives. PSI BLAST (e-value 0.5) detected 52 chains comprising five ICK, five cyclotides, seven nonknotted STPs and 35 false positives. FIG. 10 shows a comparison of the true positive hits detected in the NewNMR751 testset using different methods. Each stack portion of the bar diagram represents a different type of fold. PredSTP detected nine ICKs, five Cyclotides and six nonknotted STPs; PSI BLAST with E-value 0.01 detected 1 ICK, five Cyclotides and five nonknotted STPs; PSI BLAST with E-value 0.1 and 0.5 detected five ICKs, five Cyclotides and seven nonknotted STPs; Knoter1D detected three ICKs and five Cyclotides.

Table 6 below shows a comparison of the number of hits detected by the different tested methods in the NewNMR751 test set. Sensitivity for PredSTP and PSI BLAST was calculated based on total experimentally positive STPs (22 chains) in the NewNMR751 subset from PDB, while sensitivity for Knoter1D was calculated only for Knottins (knotted STPs).

TABLE 6

| | | | | Calculated | Calculated |
|---|---|---|---|---|---|
| | | True | False | sensitivity | precision |
| | Positive | positive | positive | (%) for | (%) for |
| Method | hits | hits | hits | STPs | STPs |
| PredSTP | 23 | 21 | 2 | 91.30 | 91.30 |
| PSI BLAST | 13 | 12 | 1 | 52.17 | 92.30 |
| with e-value | | | | | |
| 0.01 | | | | | |
| PSI BLAST | 21 | 17 | 4 | 73.90 | 80.95 |
| with e-value | | | | | |
| 0.1 | | | | | |
| PSI BLAST | 52 | 17 | 35 | 73.90 | 32.69 |
| with e-value | | | | | |
| 0.5 | | | | | |
| Knoter1D | 8 | 8 | 0 | 57.14 | 100 |

As shown in FIG. 6 and discussed above, Knoter1D detected only 8 out of 14 knotted STPs (ICKs and cyclotides) and did not detect six new ICKs as they differ significantly from the sequences of the known ICKs (knotted STPs). When PredSTP was compared with PSI-BLAST, three different E-values were used to obtain the optimum result from PSI BLAST. Among the three versions, PSI BLAST with E-value 0.1 can detect 21 chains that exhibit the highest sensitivity with a minimum number of 4 false positives. On the other hand, PredSTP detected 21 STPs including the six new ICKs missed by the detection method of Knoter 1D and PSI BLAST. Therefore, in terms of detecting all type of STPs (cyclotides, ICKs and nonknotted STPs), PredSTP demonstrates better sensitivity and precision than PSI BLAST.

The confusion matrices generated by PredSTP using the training sets Smallprotein163 and NewNMR751 subsets from PDB are shown below in Table 7.

TABLE 7

| | True | True | False | False |
|---|---|---|---|---|
| Source of data | positive | negative | positive | negative |
| Training set | 18959 | 56537 | 3463 | 1041 |
| over 200 | | | | |
| iterations | | | | |
| Smallprotein163 | 14 | 141 | 7 | 1 |
| NewNMR751 | 21 | 726 | 2 | 2 |

Table 8 below shows the comparison of evaluation matrices generated by PredSTP using the training sets Smallprotein163 and NewNMR751 subsets from PDB.

TABLE 8

| Source of data | Sensitivity | Specificity | Precision | Accuracy |
|---|---|---|---|---|
| Training set over | 94.86 | 94.11 | 84.31 | 94.30 |
| 200 iterations | | | | |
| Smallprotein163 | 93.33 | 99.29 | 66.66 | 95.09 |
| NewNMR751 | 91.30 | 99.72 | 91.30 | 99.46 |

As shown above in Table 8, PredSTP showed a better accuracy (95.09%) for Smallprotein163 than it did for the training set (94.30%), while the precision was comparatively low (66.66%). The only STP not detected (PDB id 2C4B) was a heterogenous fusion protein of an STP and a catalytically inactive variant of RNase barnase. On the other hand, a test of performance of PredSTP on the NewNMP751 subset showed an excellent accuracy (99.46%) with a better precision (90.30%) than it showed on the training set. These results indicate that PredSTP retained its performance when distinguishing STPs from out of sample cysteine rich small proteins.

### Example 2. Broader Prediction of STP Sequences

After testing the performance of PredSTP against chains from the “SmallProtein163” and “NewNMR751” subsets, which consist of sequences of similar size to the training set, PredSTP was tested against a set based on diverse taxonomy. “Eukaryota”, “Bacteria”, “Viruses”, “Archaea” and “Unassigned” subsets of proteins (see Table 2) were analyzed from the PDB. A higher proportion of STPs in eukaryotes was anticipated with respect to the total number of cysteine chains with a maximum of 75 residues and a minimum of six cysteines. There is a known paucity of disulfide bonding in bacteria and archaea compared to eukaryotes. The threshold of 75 was chosen because it is well below the length of the longest chain (86 residues long) detected as STP by PredSTP among taxonomy subsets. Table 9 below shows the discovery of STPs across major domains using the PDF protein sequence data and PredSTP.

TABLE 9

| | | | | Number of | |
|---|---|---|---|---|---|
| | Total | | Positive | proteins | |
| | # of | Total | chains | containing | Percentage |
| PDB | proteins | # of | predicted by | positive | of positive |
| subset | analyzed | chains | PredSTP | chains | chains |
| Eukaryotes | 45751 | 102748 | 636 | 139^{a} | 0.61 |
| Eubacteria | 31664 | 80664 | 3 | 2 | 0.003 |
| Archaea | 3127 | 8366 | 0 | 0 | 0 |
| Viruses | 4629 | 18642 | 4 | 3 | 0.02 |
| Unassigned | 479 | 980 | 10 | 10 | 1.02 |

^{a}For eukaryotes, 139 chains were obtained after screening 636 chains and removing those with greater than or equal to 30% sequence identity.

The percentage of positive chains in “Eukaryote” (0.61) is more than the percentage of predicted positive chains for the other three major super kingdoms. In “Eukaryotes”, 636 chains were predicted as STP positive. This number was reduced to 139 chains when chains sharing >30% sequence similarity were removed and the first 100 chains (based on PDB id) were manually cross-matched with Jmol analysis to determine true positives. This resulted in a 82% precision rate. In “Eubacteria”, “Viruses” and “Unassigned” subsets, the precisions were 50%, 33.33% and 90%, respectively, as shown in Table 10 below.

TABLE 10

| | PredSTP | True | Percent of true |
|---|---|---|---|
| | positive | (structurally) | positives |
| PDB subset | hits | positives | (precision) |
| Eukaryotes | 139 | 82 (100)^{a} | 82 |
| Bacteria | 2 | 1 | 50 |
| Archaea | 0 | 0 | NA |
| Viruses | 3 | 1 | 33 |
| Unassigned | 10 | 9 | 90 |
| Total | 115^{a} | 93 | 80.86 |

^{a}For eukaryotes, 100 of the 139 proteins were analyzed in Jmol to find true positives.

In the “Archaea” subset, PredSTP did not predict any potential STP toxins, resulting in no precision. In total, 115 positive hits were analyzed from the “Taxonomy” subset and 93 chains were found as true positive with an overall 80.86% precision. Individual precision rates for bacteria and viruses were low; this is potentially an artifact of their small sizes. In addition, some bacteria may contain iron-sulfur like transport proteins that mimic STPs by primary structure but are functionally distinct. The number of protein chains containing a minimum of six cysteines and consisting of a maximum 75 residues were also calculated for the same taxonomy subsets from PDB, and the percentages of predicted STPs were 30.08, 6.66, 0, 14.81 and 47.61 for Eukaryotes, bacteria, archaea, virus and unassigned, respectively, as shown in Table 11 below.

TABLE 11

| | | | | | Percent of | Percent of |
|---|---|---|---|---|---|---|
| | | | Type | Type | predicted | predicted |
| | Total # | | 1 | 2 | STPs in type | STPs in type |
| PDB subset | of chains | PredSTP | chain | chain | 1 chains | 2 chains |
| Eukaryotes | 102748 | 636 | 2114 | 32348 | 30.08 | 1.96 |
| Bacteria | 90664 | 3 | 45 | 9294 | 6.66 | 0.03 |
| Archaea | 8366 | 0 | 6 | 663 | 0.00 | 0 |
| Virus | 18642 | 4 | 27 | 3477 | 14.81 | 0.11 |
| Unassigned | 980 | 10 | 21 | 43 | 47.61 | 23.25 |

After testing protein chains from different organismal taxonomy subsets in PDB, it was observed that only 6.66% and 0% of chains possessing a minimum of six cysteines and maximum 75 residues were predicted as STPs in bacteria and archaea, respectively, as shown in Table 11. In contrast, 30% of the small cysteine-containing chains were predicted as STPs in eukaryotes.

### Example 3. Predicting STP and NTP Sequences

Methods used for the prediction of compact NTP sequences in addition to STP sequences were similar to those set forth in Examples 1-2 above. For the training data set, the known STP sequence collection, a total of 108 sequences were retained from the knottin database set and 36 sequences were from the PDB set, leaving 144 canonical STPs. For the control negative sequence collection, sequences classified as negative control were collected from PDB, using a criterion that was species agnostic and stipulated a solved X-ray crystallography structure of less than 1.5 Å diffraction and having a sequence less than 150 residues. Sequences containing tri-disulfide bonds with an average distance less than 1 nm were omitted. The resulting set candidate sequences was reduced to remove redundant sequences sharing ≧40% sequence identity using CD-HIT. A remaining 442 sequences were classified as non-STP sequences for the purposes of this example.

Different feature sets were considered, similar to those set out in Table 1 above but eliminating those relating to Table 1's Feature Set 4, which included presence of amino acid between C4-C5 and C5-C6 and presence of double consecutive cysteines in the sequence in the 4^{th} and 5^{th} loop. Again, a Support Vector Machine (SVM) classifier/predictor implementation was used to elucidate STP toxins. The SVM was implemented using the e1071 library in R (2.15.1). Sensitivity, specificity, precision and accuracy were determined after ten-fold cross validation for the feature sets. Initial gamma and cost were set to 0.01 and 0.1, respectively, with the best output at 0.0087. Given 145 knottin and 445 nonknottin chains, 50 and 150 random samples were chosen, respectively for a test set over 10 iterations. Feature sets were prioritized based on accuracy.

STP sequences were predicted from the test sets described previously (Table 1) using the feature set that included Normalized Bonding Distance (NBD) between C1-C4, C2-C5 and C3-C6, Calculating the number of occurrences of each of the amino acids, the aggregate number of occurrences of hydrophobic (F,Y,L,I,A,M,C,W,V), hydrophilic (R,K,N,D,A,P), and neutral (G,H,S,T,Q) amino acids, and plus the total peptide length and the least loop length ratio. Ultimately, the model with this feature set was used for the basis of the remainder of the example because (i) it had slightly higher accuracy and (ii) it contained higher feature dimensionality, therefore offering potentially higher discrimination.

Due to the limited throughput of the Knotter1D interface, only the same “NewNMR751” and “Smallprotein163” subsets were used as in Examples 1-2 above. Results from only the eukaryotic test sets were filtered to remove sequences with ≧30% chain identity and compared against Jmol analysis. Chains with three disulfide bonds in close proximity and exhibiting canonical STP connectivity (C1-C4, C2-C5, C3-C6) were initially considered as true positives. True positives were further cross matched with their PDB annotations to make the final confirmation.

The SmallProtein163 data subset from PDB was analyzed to determine potential automated knottin classification. From this subset, the PredSTP model was able to identify 43 of the 163 potential chains, representing 31 of the 92 proteins, as STPcontaining. These putative STP structures were verified by examining their disulfide bonding patterns in Jmol. Of the 31 identified proteins, PDB classified 12 of them as STP. An analysis of the 119 negative knottin chains predicted by the algorithm, representing 62 proteins, demonstrated no false negative. Overall, 12 of 12 STPs were correctly identified from this diverse set of protein classes and families. The sensitivity, specificity, precision and accuracy for this particular dataset were 100%, 76.22%, 38.70% and 88.2%, respectively (Table 4). Among the 19 false positives, 11 contain a compact tri-disulfide (compact NTP category) fold possessing functional activity like STPs. Thus, the algorithm also successfully predicted compact NTP sequences. When using the algorithm to predict both STP and compact NTP sequences, the precision goes up to 74.19%.

The NewNMR751 subset was also tested by PredSTP. The model detected 41 chains from 41 different proteins. Analyzing the disulfide connectivity of the positive hits by Jmol, 23 chains were confirmed as true positive. Based on the number of true positives over false positives, the calculated precision was 56.09%. The true positive chains were further classified into 9 ICKs, 5 cyclotides and 9 nonknotted STPs. This same set was also analyzed by, PSI BLAST and Knoter1D and 20 and 8 were detected as positive hits, respectively. More descriptively, Knoter1D detected all 5 cyclotides, 3 of the 9 ICKs and none of the nonknotted STPs. PSI-BLAST was applied to observe STP sequences with lower similarity and detected 4 ICKs, 5 cyclotides, 6 nonknotted and 5 were false positive. Functionally, 10 of the 41 predicted chains were antimicrobial peptides and 18 of them were other toxins. The remainder demonstrated diverse functions, often associated with STP toxins. An analysis of the false positives again found 12 out of 17 false positives possessing the NTP tri-disulfide array exhibiting the typical functional properties as STPs. Using the algorithm to predict STPs and compact NTPs increases the precision to 87.50%.

### REFERENCES

The following publications are hereby incorporated by reference.
Matsumura M, Signor G, Matthews B W. Substantial increase of protein stability by multiple disulphide bonds. Nature. 1989; 342:291-3.
Gracy J, Le-Nguyen D, Gelly J-C, Kaas Q, Heitz A, Chiche L. KNOTTIN: the knottin or inhibitor cystine knot scaffold in 2007. Nucleic Acids Res. 2008; 36 (Database issue): D314-319.
Conibear A C, Rosengren K J, Daly N L, Henriques S T, Craik D J. The cyclic cystine ladder in θ-defensins is important for structure and stability, but not antibacterial activity. J Biol Chem. 2013; 288:10830-40.
Gelly J-C, Gracy J, Kaas Q, Le-Nguyen D, Heitz A, Chiche L. The KNOTTIN website and database: a new information system dedicated to the knottin scaffold. Nucleic Acids Res. 2004; 32(Database issue):D156-159.
Kedarisetti P, Mizianty M J, Kaas Q, Craik D J, Kurgan L. Prediction and characterization of cyclic proteins from sequences in three domains of life. Biochim Biophys Acta. 2014; 1844(1 Pt B):181-90.
Mulvenna J P, Wang C, Craik D J. CyBase: a database of cyclic protein sequence and structure. Nucleic Acids Res. 2006; 34(Database issue): D192-194.
Wang C K L, Kaas Q, Chiche L, Craik D J. CyBase: a database of cyclic protein sequences and structures, with applications in protein discovery and engineering. Nucleic Acids Res. 2008; 36(Database issue):D206-210.
Kaas Q, Yu R, Jin A-H, Dutertre S, Craik D J. ConoServer: updated content, knowledge, and discovery tools in the conopeptide database. Nucleic Acids Res. 2012; 40(Database issue): D325-330.
Herzig V, Wood D L A, Newell F, Chaumeil P-A, Kaas Q, Binford G J, Nicholson G M, Gorse D, King G F. ArachnoServer 2.0, an updated online resource for spider toxin sequences and structures. Nucleic Acids Res. 2011; 39(Database issue):D653-657.
Muggleton S, King R D, Sternberg M J. Protein secondary structure prediction using logic-based machine learning. Protein Eng. 1992; 5:647-57.
Bock J R, Gough D A. Predicting protein—protein interactions from primary structure. Bioinform Oxf Engl. 2001; 17:455-60.
Cai C Z, Han L Y, Ji Z L, Chen X, Chen Y Z. SVM-Prot: Web-based support vector machine software for functional classification of a protein from its primary sequence. Nucleic Acids Res. 2003; 31:3692-7.
Hua S, Sun Z. A novel method of protein secondary structure prediction with high segment overlap measure: support vector machine approach. J Mol Biol. 2001; 308:397-407.
Cai Y D, Liu X J, Xu X, Zhou G P. Support vector machines for predicting protein structural class. BMC Bioinformatics. 2001; 2:3.
Hua S, Sun Z. Support vector machine approach for protein subcellular localization prediction. Bioinformatics. 2001; 17:721-8.
Huang Y, Niu B, Gao Y, Fu L, Li W. C D-HIT Suite: a web server for clustering and comparing biological sequences. Bioinformatics. 2010; 26:680-2.
Li W, Godzik A. Cd-hit: a fast program for clustering and comparing large sets of protein or nucleotide sequences. Bioinformatics. 2006; 22:1658-9.
Altschul S F, Madden T L, Schaffer A A, Zhang J, Zhang Z, Miller W, Lipman D J. Gapped BLAST and PSI-BLAST: a new generation of protein database search programs. Nucleic Acids Res. 1997; 25:3389-402.

What is claimed is:

1. A method for identifying peptide sequences including sequential tri-disulfide peptide (STP) structures using a Support Vector Machine (SVM)-based model, comprising:
obtaining a set of training peptide sequences, wherein the training peptide sequences are identified as containing STP structures or lacking STP structures;
identifying a numerical order of six cysteines in each training peptide sequence which is C1-C2-C3-C4-C5-C6;
extracting a set of features from the training peptide sequences, wherein the set of features comprises three Normalized Bonding Distance (NBD) values, a presence of double consecutive cysteines in a C4-C5 loop, a presence of double consecutive cysteines in a C5-C6 loop, a least loop length to total length ratio, a total number of amino acid residues in the sequence, an aggregate number of occurrences of cysteine, serine, arginine, histindine, lysine (C,S,R,H,K), an aggregate number of occurrences of hydrophobic (F,Y,L,I,A,M,C,W,V) amino acids, an aggregate number of occurrences of hydrophilic (R,K,N,D,A,P) amino acids, and an aggregate number of occurrences of neutral (G,H,S,T,Q) amino acids;
compiling the features into a feature matrix used for training the SVM-based model to predict presence of STP structures;
obtaining an unknown peptide sequence;
identifying a numerical order of six cysteines in the unknown peptide sequence which is C1-C2-C3-C4-C5-C6;
extracting the set of features from the unknown peptide sequence; and
using the SVM-based model to analyze the features of the unknown peptide sequence in relation to the feature matrix and to identify whether the unknown peptide sequence includes a STP structure.

2. The method of claim 1, wherein the three Normalized Bonding Distance (NBD) values are extracted by using the following equations:
NBD₁=100/(|P₁−P̅₁|+10)
NBD₂=100/(|P₂−P̅₂|+10)
NBD₃=100/(|P₃−P̅₃|+10)
wherein P₁=ΔC_{1,4}, P₂=ΔC_{2,5}, P₃=ΔC_{3,6}, P̅₁=x̅ΔC_{1,4}, P̅₂=x̅ΔC_{2,5}, and P̅₃=x̅ΔC_{3,6}.

3. The method of claim 1, wherein the least loop length to total length ratio is extracted by calculating min(ΔC_{i,i+1}) divided by the total length of the sequence, and wherein if min(ΔC_{i,i+1}) is more than 3, then the value for the feature is 0.

4. The method of claim 1, wherein the unknown peptide sequence is obtained by searching a genome.

5. The method of claim 1, wherein the unknown peptide sequence is an artificial sequence.

6. A method for identifying peptide sequences including compact stabilized tri-disulfide peptide structures using a Support Vector Machine (SVM)-based model, comprising:
obtaining a set of training peptide sequences, wherein the training peptide sequences are identified as containing compact stabilized tri-disulfide peptide structures or lacking compact stabilized tri-disulfide peptide structures;
identifying a numerical order of six cysteines in each training peptide sequence which is C1-C2-C3-C4-C5-C6;
extracting a set of features from the training peptide sequences, wherein the set of features comprises three Normalized Bonding Distance (NBD) values, a least loop length to total length ratio, a total number of amino acid residues in the sequence, a total number of occurrences of each amino acid in the sequence, an aggregate number of occurrences of hydrophobic (F,Y,L,I,A,M,C,W,V) amino acids, an aggregate number of occurrences of hydrophilic (R,K,N,D,A,P) amino acids, and an aggregate number of occurrences of neutral (G,H,S,T,Q) amino acids;
compiling the features into a feature matrix used for training the SVM-based model to predict presence of compact stabilized tri-disulfide peptide structures;
obtaining an unknown peptide sequence;
identifying a numerical order of six cysteines in the unknown peptide sequence which is C1-C2-C3-C4-C5-C6;
extracting the set of features from the unknown peptide sequence; and
using the SVM-based model to analyze the features of the unknown peptide sequence in relation to the feature matrix and to predict whether the unknown peptide sequence includes a compact stabilized tri-disulfide peptide structure.

7. The method of claim 6, wherein the three Normalized Bonding Distance (NBD) values are extracted by using the following equations:
NBD₁=100/(|P₁−P̅₁|+10)
NBD₂=100/(|P₂−P̅₂|+10)
NBD₃=100/(|P₃−P̅₃|+10)
wherein P₁=ΔC_{1,4}, P₂=ΔC_{2,5}, P₃=ΔC_{3,6}, P̅₁=x̅ΔC_{1,4}, P̅₂=x̅ΔC_{2,5}, and P̅₃=x̅ΔC_{3,6}.

8. The method of claim 6, wherein the least loop length to total length ratio is extracted by calculating min(ΔC_{i,i+1}) divided by the total length of the sequence, and wherein if min(ΔC_{i,i+1}) is more than 3, then the value for the feature is 0.

9. The method of claim 1, wherein the unknown peptide sequence is obtained by searching a genome.

10. The method of claim 1, wherein the unknown peptide sequence is an artificial sequence.
